(19) United States
(12) Patent Application Publication
(10) Pub. No.: US 2020/0330364 A1
(43) Pub. Date: Oct. 22, 2020

(54) INSECT REPELLENT COMPOSITIONS AND METHODS OF USE

(71) Applicant: SMOKY VALLEY LOTIONS & SOAPS, Waimea, HI (US)

(72) Inventor: Jo King, Waimea, HI (US)

(21) Appl. No.: 16/763,493

(22) PCT Filed: Nov. 13, 2018
(86) PCT No.: PCT/US2018/060591

§ 371 Date: May 12, 2020

Related U.S. Application Data
(60) Provisional application No. 62/585,032, filed on Nov. 13, 2017.

(51) Int. Cl. A61K 8/9789; A01N 65/26; A61K 8/67; A61K 8/81; A61K 8/36; A61K 8/92; A61Q 17/02; A61Q 19/00; A61Q 17/04

(52) U.S. Cl. CPC A61K 8/9789; A01N 65/26; A61K 8/678; A61K 8/8147; A61Q 17/04; A61K 8/922; A61Q 17/02; A61Q 19/004; A61K 8/36

(57) ABSTRACT

The disclosure relates generally to insect repellent compositions and methods for using the same.

### CROSS-REFERENCE TO RELATED APPLICATION(S)

This application is a 35 U.S.C. § 371 U.S. National Stage Application of International Application No. PCT/US2018/060591, filed Nov. 13, 2018, which claims priority to U.S. Provisional Patent Application Ser. No. 62/585,032, filed Nov. 13, 2017, the entire contents of which is incorporated herein by reference.

### TECHNICAL FIELD

The present disclosure provides insect repellent compositions containing neem and methods for using the same.

### BACKGROUND

Insect repellents are used to protect individuals from insect bites and symptoms resulting from contact with insects, including numerous insect-borne diseases such as malaria, Lyme disease, West Nile fever, and Zika virus. Insects that often serve as carriers for disease include fleas, flies, ticks, and mosquitos. N,N-Diethyl-meta-toluamide (DEET) is the most common active ingredient in insect repellents. However, for some individuals DEET is an irritant that can cause unwanted epidermal reactions, difficulty breathing, burning eyes and headaches. DEET-based products have also been found to have toxicities for animals, including fish.

As such, there remains a need for effective insect repellents that are safe for users and the environment.

### SUMMARY

In some aspects provided herein are compositions comprising up to about 40% neem, about 3% kokum butter, an antioxidant, an emulsifying agent and a preservative. In some aspects provided herein are compositions comprising neem, taro, a fragrance, kokum butter, an antioxidant, a preservative, and an emulsifying agent. In some aspects provided herein are compositions comprising neem, taro, gardenia, kokum butter, vitamin E, Optiphen PLUS, and ECOMulse or neem, kokum butter, cinnamon essential oil, fragrance, sorbic acid, and ECOMulse. In some aspects provided herein are compositions comprising, on a weight basis, about 7% neem, about 2.5% taro, about 2.5% gardenia, and further comprising kokum butter, vitamin E, Optiphen PLUS, and ECOMulse. In some aspects provided herein are compositions comprising, on a weight basis, about 25% neem, about 3% kokum, sorbic acid, and ECOMulse, and a fragrance.

In some aspects provided herein are sunscreens, bug repellents, body salves, body creams, shampoos, conditioners, soaps, candles, incenses, diffusers, body sprays, toilet sprays, animal sprays, and/or plant sprays composition comprising neem, a butter, an antioxidant, an emulsifying agent and a preservative. In some aspects provided herein are sunscreens, bug repellants, body salves, body creams, shampoos, conditioners, soaps, candles, incenses, diffusers, body sprays, toilet sprays, animal sprays, and/or plant sprays composition comprising neem, kokum butter, and an ECOMulse and optionally further comprises one or more of taro, a fragrance, Optiphen PLUS, sorbic acid, and/or vitamin E.

In some aspects provided herein are methods of repelling insects from a subject comprising administering to skin of the subject, for example a child, an effective amount of any of the compositions disclosed and described herein.

In some aspects provided herein are methods of preventing and/or treating sunburn from a subject comprising administering to skin of the subject, for example a child, an effective amount of the compositions disclosed and described herein.

In some aspects provided herein are methods of repelling insects and preventing and/or treating sunburn from a subject, for example a child, comprising administering to skin of the subject an effective amount of the compositions disclosed and described herein.

In some embodiments, the neem is present in an amount of from about 1% to about 20%, by weight. In some embodiments, the neem is present in an amount of less than 10%, by weight. In some embodiments, the antioxidant is vitamin E and/or tocopherol. In some embodiments, the antioxidant is present in an amount of between about 0.01% and 5%, by weight. In some embodiments, the emulsifying agent is carbopol (Carbomer) 940, carbopol 934, carbopol 941, carbopol 1342 and gulf Polymer P18 (octadecene/maleic anhydride copolymer), a C12-C22 alkyl-substituted acrylic acid copolymer, or any combination thereof. In some embodiments, the emulsifying agent is present in an amount of between about 1% and 10%, by weight. In some embodiments, the preservative is methylparaben, propylparaben, benzyl alcohol, ascorbyl palmitate, ascorbic acid, or any combination thereof. In some embodiments, the preservative comprises phenoxyethanol, caprylyl glycol, sorbic acid, or any combination thereof. In some embodiments, the preservative is Optiphen PLUS or sorbic acid. In some embodiments, the preservative is present in an amount of about 0.01% and 5%, by weight.

In some embodiments, the composition further comprises taro. In some embodiments, the taro is present in an amount of between about 0.1% to about 5%, by weight.

In some embodiments, the composition further comprises a scent agent. In some embodiments, the scent agent is gardenia or cinnamon essential oil.

In some embodiments, the composition is free or substantially free of deet, citronella, and equivalents or combinations thereof.

In some embodiments, the composition is a topical composition. In some embodiments, the topical composition is in the form of a cream.

In some embodiments, the composition is co-administered with a corticosteroid selected from the group consisting of clobetasol diproprionate, betamethasone diproprionate, halbetasol proprionate, diflorasone diacetate, fluocinonide, halcinonide, amcinonide, desoximetasone, triamcinolone acetonide, mometasone furoate, fluticasone proprionate, fluocinolone acetonide, hydrocortisone valerate, hydrocortisone butyrate, triamcinalone acetonide, desonide, prednicarbate, prednisolone, methylprednisolone, dexamethasone, naflocort, deflazacort, halopredone acetate, budesonide, beclomethasone dipropionate, hydrocortisone, clocortolone pivalate, methylprednisolone aceponate, dexamethasone palmitoate, tipredane, hydrocortisone aceponate, alclometasone dipropionate, halometasone, methylprednisolone suleptanate, rimexolone, prednisolone farnesylate, ciclesonide, deprodone propionateloteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and combinations thereof.

In some embodiments, the composition is co-administered with a further agent selected from the group consisting of immunomodulators, antibiotics, immunosuppressants, and anti-itch drugs.

### BRIEF DESCRIPTION OF THE DRAWINGS

FIG. 1 shows mean protection times in cage tests with Ae. aegypti. The x-axis shows the bite incidents, the y-axis gives the mean protection times in hours (±standard deviation, SD). Left column: time until first bite; right column: time until first confirmed bite (FCB). Means were generated from data sets provided by 3 volunteers (n=3).

### DETAILED DESCRIPTION

The present disclosure is directed to methods for repelling insects and preventing insect-borne diseases and compositions for use in these methods.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a preservative” includes a plurality of preservatives.

As used herein the following terms have the following meanings:

The term “about” when used before a numerical designation, e.g., temperature, time, amount, concentration, and such other, including a range, indicates approximations which may vary by (+) or (−) 20%, 10%, 5% or 1%.

The terms “administering,” “administer” and the like refer to introducing an agent (e.g., repellent) into a subject. Typically, an effective amount is administered. Any route of administration, such as topically, can be used. The terms and phrases “administering” and “administration of,” when used in connection with a composition (and grammatical equivalents) refer both to direct administration, which may be administration to a subject by a medical professional or by self-administration by the subject, and/or to indirect administration, which may be the act of prescribing. “Periodic administration” or “periodically administering” refers to multiple treatments that occur on a daily, weekly, or a monthly basis. Periodic administration may also refer to administration of an agent one, two, three or more time(s) per day. “Co-administration” refers to administration of two or more compositions to the same subject. Co-administration may be simultaneous or at about the same time or may be sequentially.

An “effective amount” is an amount of an agent or compound (e.g., repellent) sufficient to effect beneficial or desired results. An effective amount can be in one or more administrations, applications or dosages. Determination of these parameters is well within the skill of the art. These considerations, as well as effective formulations and administration procedures are well known in the art and are described in standard textbooks.

A “subject” or “individual” is used interchangeably herein and refers to a vertebrate, for example a primate, a mammal or preferably a human. Mammals include, but are not limited to equines, canines, bovines, ovines, murines, rats, simians, humans, farm animals, and pets. In some embodiments, the human is an infant, a toddler, a child, or the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Compositions and Formulations

Insect repellents are used to displace and/or kill insects. In the earlier 1950s, the insect repellent properties of N,N-diethyl-m-toluamide (DEET) were discovered and the first DEET product was introduced commercially by the mid-1950s. DEET is still the most widely used mosquito repellent and while it is generally regarded as safe, toxic effects of DEET have been recorded, including encephalopathy in children, urticaria syndrome, anaphylaxis, hypotension and decreased heart rate.

In some embodiments, the compositions comprise neem. Neem, also called Azadirachta indica is an aboriginal tree found in tropical and semi-tropical countries like Burma and India. Neem is considered non-toxic to humans and various parts of the tree are used as active ingredients in different industries.

In some embodiments, the neem is present in an amount of from about 1% to about 40%, about 1% to about 30%, about 1% to about 20%, from about 2% to about 15%, from about 3% to about 12%, from about 5% to about 10%, or from about 7% to about 9%, by weight. In some embodiments, the neem is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, or about 40% by weight. In some embodiments, the neem is present in an amount of less than about 10%, about 9%, about 8%, about 7%, about 6%, about 5%, about 4%, about 3%, about 2%, or about 1%, by weight.

In some embodiments the composition comprises a butter. Butters can be used to moisturize and rejuvenate dry skin. Non-limiting examples of suitable butters include, kokum butter, cocoa butter, avocado butter, mango butter, olive butter, hemp seed butter, shea butter, and almond butter. In some embodiments the butter is kokum butter.

In some embodiments, the butter is present in an amount of from about 1% to about 20%, from about 2% to about 15%, from about 3% to about 10%, or from about 4% to about 8%, by weight. In some embodiments, the butter is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, by weight. In some embodiments, the butter is present in an amount of less than about 10%, about 9%, about 8%, about 7%, about 6%, about 5%, about 4%, about 3%, about 2%, or about 1%, by weight.

In some embodiments, the composition comprises an antioxidant. Any antioxidant known to one of skill in the art is suitable. In some embodiments, the antioxidant is vitamin E and/or tocopherol.

In some embodiments, the antioxidant is present in an amount of between about 0.01% to about 5%, about 0.1% to about 1%, or about 0.2% to about 0.75%, by weight. In some embodiments, the antioxidant is present in an amount of about 0.01%, about 0.025%, 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, or about 5%, by weight.

In some embodiments, the composition comprises an emulsifying agent. Any emulsifying agent known to one of skill in the art is suitable. In some embodiments, the antioxidant is carbopol (Carbomer) 940, carbopol 934, carbopol 941, carbopol 1342 and gulf Polymer P18 (octadecene/maleic anhydride copolymer), a C₁₂-C₂₂ alkyl-substituted acrylic acid copolymer, and any combination thereof.

In some embodiments, the emulsifying agent is present in an amount of between about 1% and about 10%, about 2% and about 8%, or about 3% and about 6%, by weight. In some embodiments, the emulsifying agent is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, by weight.

In some embodiments, the composition comprises a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea. In some embodiments, the preservative is methylparaben, propylparaben, benzyl alcohol, ascorbyl palmitate, ascorbic acid, or any combination thereof. In some embodiments, the preservative comprises phenoxyethanol, caprylyl glycol, and sorbic acid. In some embodiments, the preservative is Optiphen PLUS.

In some embodiments, the preservative and/or a stabilizer is present in an amount of between about 0.01% and about 5%, about 2% and about 8%, or about 3% and about 6%, by weight. In some embodiments, the emulsifying agent is present in an amount of between about 0.01% to about 5%, about 0.1% to about 1%, or about 0.2 to about 0.75%, by weight. In some embodiments, the antioxidant is present in an amount of about 0.01%, about 0.025%, 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, or about 5%, by weight.

In some embodiments, the composition further comprises taro. In some embodiments, the taro is present in an amount of between about 0.01% to about 5%, about 0.1% to about 1%, or about 0.2% to about 0.75%, by weight. In some embodiments, the taro is present in an amount of about 0.01%, about 0.025%, 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, or about 5%, by weight.

In some embodiments, the composition further comprises a scent. Any scent known to one of skill in the art is suitable including, for example, citrus, gardenia, heliotrope, hyacinth, honeysuckle, jasmin, jonquil, lavender, lavandin. lilac, lily-of-the-valley, mimosa, acacia, orange, rose, rose oxide, rosemary, violet, etc. Spicy fragrances can be bisal, bay, birch tar, caraway, cinnamon, cedar leaf, clove, clover, musk, nutmeg, oakmoss, orris root, sage, sweet grass, tuberose, tonka, vanillin, ethyl vanillin, benzyl alcohol, ambrettolide, galaxolide, geraniol, hexadecanolide, indole, albdanum, lemon grass, neroli, narcissus, petigrain resida, and ambergris fixative. In some embodiments, the scent is gardenia. In some embodiments, the scent is not an essential oil.

In some embodiments, the scent is present in an amount of between about 0.01% to about 5%, about 0.1% to about 1%, or about 0.2% to about 0.75%, by weight. In some embodiments, the scent is present in an amount of about 0.01%, about 0.025%, 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, or about 5%, by weight.

In some embodiments, the composition is free or substantially free of deet, citronella, and equivalents or combinations thereof. In some embodiments, the composition is non-toxic.

In some embodiments, the composition comprises neem, taro, gardenia, kokum butter, an antioxidant, a preservative, and an emulsifying agent. In some embodiments, the composition comprises neem, taro, gardenia, kokum butter, vitamin E, Optiphen PLUS, and ECOMulse. In some embodiments, composition comprises about 7% neem, about 2.5% taro, about 2.5% gardenia, about kokum butter, vitamin E, Optiphen PLUS, and ECOMulse.

In some embodiments, the composition is a sunscreen, bug repellant, body salve, body cream, shampoo, conditioner, soap, candle, incense, diffuser, body spray, toilet spray, animal spray, and/or plant spray comprising neem, a butter, an antioxidant, an emulsifying agent and a preservative. In some embodiments, the sunscreen, bug repellant, body salve, body cream, shampoo, conditioner, soap, candle, incense, diffuser, body spray, toilet spray, animal spray, and/or plant spray composition comprises neem, taro, gardenia, kokum butter, vitamin E, Optiphen PLUS, and an ECOMulse.

TABLE 1
Formulation 1

| | Formulation 1 |
|---|---|
| | wt % |
| neem | 7.04% |
| taro | 2.5% |
| gardenia | 2.5% |
| kokum butter | 3.79% |
| vitamin E/mixed tocopherols | 0.5% |
| OptiPhen Plus | 0.75% |
| Ecomulse | 4% |
| Water | up to 100% |
| TOTAL | 100.00% |

TABLE 2
Formulation 2

| | Formulation 2 |
|---|---|
| | wt % |
| neem | 25% |
| fragrance | 1.5-3% |
| kokum butter | 3% |
| cinnamon essential oil | 0.5% |
| sorbic acid | 0.75% |
| Ecomulse | 5.0-6.0% |
| Water | up to 100% |
| TOTAL | 100.00% |

TABLE 3
Formulation 3

| | Formulation 3 |
|---|---|
| | wt % |
| neem | up to 50% |
| taro (Kale'ai (taro water)) | up to 30% |
| Lye | up to 35% |
| fragrance | up to 1% |

one or more of Castor Oil, Palm
Kernel oil, Palm oil, Mango Butter,
Kokum Butter, Vit E, Grapeseed oil,
Cocoa Butter, Avocado Oil

| | |
|---|---|
| Water | up to 100% |
| TOTAL | 100.00% |

TABLE 4
Formulation 4

| | Formulation 4 |
|---|---|
| | wt % |
| neem | up to 75% |
| beeswax | up to 25% |
| isopropyl myristate | up to 5% |
| fragrance | up to 2% |
| vit E | up to 1% |
| Water | up to 100% |
| TOTAL | 100.00% |

TABLE 5
Formulation 5

| | Formulation 5 |
|---|---|
| | wt % |
| neem | up to 75% |
| beeswax | up to 25% |
| Vit e | up to 1% |
| fragrance | up to 2% |
| TOTAL | 100.00% |

TABLE 6
Formulation 6

| | Formulation 6 |
|---|---|
| | wt % |
| Kale'ai (taro water) | up to 97% |
| neem | up to 5% |
| preservative | up to 1% |
| fragrance | up to 5% |
| TOTAL | 100.00% |

The composition may comprise an excipient, a salt, diluents, carriers, vehicles and such other inactive agents well known to the skilled artisan. Vehicles and excipients can include, for example, talc, gum Arabic, lactose, starch, magnesium stearate, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Compositions may be prepared using conventional techniques that may include sterile isotonic saline, water, 1,3-butanediol, ethanol, 1,2-propylene glycol, polyglycols mixed with water, Ringer's solution, etc. In one embodiment, a coloring agent is added to facilitate in locating and properly placing the composition to the intended site.

In some embodiments, the compositions further comprise a corticosteroid, immunomodulators, antibiotics, immunosuppressants, anti-itch drugs, or any combination thereof. Suitable corticosteroids include, for example, clobetasol diproprionate, betamethasone diproprionate, halbetasol proprionate, diflorasone diacetate, fluocinonide, halcinonide, amcinonide, desoximetasone, triamcinolone acetonide, mometasone furoate, fluticasone proprionate, fluocinolone acetonide, hydrocortisone valerate, hydrocortisone butyrate, triamcinalone acetonide, desonide, prednicarbate, prednisolone, methylprednisolone, dexamethasone, naflocort, deflazacort, halopredone acetate, budesonide, beclomethasone dipropionate, hydrocortisone, clocortolone pivalate, methylprednisolone aceponate, dexamethasone palmitoate, tipredane, hydrocortisone aceponate, alclometasone dipropionate, halometasone, methylprednisolone suleptanate, rimexolone, prednisolone farnesylate, ciclesonide, deprodone propionateloteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and combinations thereof.

In some embodiments, the compositions further comprise a waterproofing agent. Suitable waterproofing agents include, but are not limited to, C30-C38 olefin/isopropyl maleate/MA copolymer, an acrylate copolymer, poly(vinylpyrrolidone/eicosene)copolymer, a silicone (e.g., cyclopentasiloxane), and the like.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition can be formulated as a liquid, a lotion, a spray, an aerosol lotion, a cream, a gel, an ointment, and the like. In some embodiments, the composition is a lotion or a cream.

In some embodiments, the composition is a sunscreen, a bug repellant, a body salve, a body cream, shampoo (including pet shampoo), conditioner, soap (e.g., liquid and hard), candle, incense, diffuser (including in a clip-on apparatus), body spray, toilet spray, animal spray (e.g., horse spray), and/or plant spray.

In some embodiments, the composition further comprises oil of lemon eucalyptus PMD (Citriodiol.Cis and Trans p-menthane-3,8-diol). In some embodiments the oil of lemon eucalyptus PMD comprises about 1%, about 2%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, or more, by weight, of the composition. In some embodiments, the composition comprises no or substantially no oil of lemon eucalyptus.

### Methods of Use

The compositions of the present disclosure are useful for repelling insects and/or as a sunscreen, bug repellant, body salve, body cream, shampoo, conditioner, soap, candle, incense, diffuser, body spray, toilet spray, animal spray, and/or plant spray.

In some embodiments, the methods comprise administering to skin of the subject an effective amount of a composition disclosed and described herein. In some embodiments, the methods provide prevention and/or treatment of sunburn from a subject comprising administering to skin of the subject an effective amount of a composition disclosed and described herein.

In some embodiments, the composition causes no or substantially no adverse epidermal reaction, difficulty breathing, burning eyes, headaches, and/or the like in a subject administered the composition as compared to a control composition, for example, a composition comprising DEET. In some embodiments, the composition causes no or substantially no toxicities for animals (e.g., fish), as compared to a control composition, for example, a composition comprising DEET.

In some embodiments, the composition causes a reduction or lessened degree of adverse epidermal reactions, difficulty breathing, burning eyes, headaches, and/or the like in a subject administered the composition as compared to a control composition, for example, a composition comprising DEET. In some embodiments, the composition causes a reduction or lessened degree of toxicities for animals (e.g., fish), as compared to a control composition, for example, a composition comprising DEET.

The compositions can be administered alone or in combination with a second composition. A non-limiting example of a suitable second composition is a corticosteroid, immunomodulators, antibiotics, immunosuppressants, and anti-itch drugs. Suitable corticosteroids include, for example, clobetasol diproprionate, betamethasone diproprionate, halbetasol proprionate, diflorasone diacetate, fluocinonide, halcinonide, amcinonide, desoximetasone, triamcinolone acetonide, mometasone furoate, fluticasone proprionate, fluocinolone acetonide, hydrocortisone valerate, hydrocortisone butyrate, triamcinalone acetonide, desonide, prednicarbate, prednisolone, methylprednisolone, dexamethasone, naflocort, deflazacort, halopredone acetate, budesonide, beclomethasone dipropionate, hydrocortisone, clocortolone pivalate, methylprednisolone aceponate, dexamethasone palmitoate, tipredane, hydrocortisone aceponate, alclometasone dipropionate, halometasone, methylprednisolone suleptanate, rimexolone, prednisolone farnesylate, ciclesonide, deprodone propionateloteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and combinations thereof.

In some embodiments, the compositions are applied at least once daily, at least twice daily, at least three times daily, at least four times daily, at least five times daily, at least six times daily, at least seven times daily, at least eight times daily, at least nine times daily, at least ten times daily, or more. In some embodiments, the compositions are applied less than ten times daily, nine times daily, eight times daily, seven times daily, six times daily, five times daily, four times daily, three times daily, twice daily, or once daily.

In some embodiments, the compositions provide protection (i.e., from insects and/or sun) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, or more. In some embodiments, the compositions provide complete or substantially complete protection (i.e., from insects and/or sun) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, or more.

In some embodiments, the compositions provide protection (i.e., from insects and/or sun) for at least about 5 minutes, at least about 10 minutes, at least about 20 minutes, at least about 30 minutes, at least about 45 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, or more compared to protection provided by a conventional insect repellent. Non-limiting examples of conventional insect repellents include repellents containing deet, picaridin, IR3535, 2-Undecanone, oil of lemon eucalyptus, or any combination thereof.

In some embodiments, the composition provide protection against a disease (e.g., reduce spread and/or occurrence of a disease) caused by a mosquito, a fly, a flea, a louse, a moth, a beetle, a tick, a midge, a sandfly, or the like. Non-limiting examples of diseases include dengue fever, Chagas disease, babesiosis, lyme disease, malaria, plague, leishmaniasis, Rocky Mountain spotted fever, West Nile, Zika, African trypanosomiasis, yellow fever, and the like.

### EXAMPLES

### Example 1: Bite-Preventing Efficacy of Neem Repellent Cream

### Materials and Methods.

Test Formulations & Application.

A sample of a mosquito repelling cream containing 30% Neem was stored in its original packaging at 20-22° C. until the start of the test. Prior to the application, the skin on the forearm was washed with fragrance-free soap, rinsed with water and wiped with 50% isopropyl alcohol. The cream was shaken well and applied in a thick layer to the skin on the forearm of each volunteer. Shortly after the application, the first efficacy test was conducted.

Test Mosquitos (Aedes aegypti).

Female mosquitos of the genus Aedes were reared according to the standard protocol at a temperature of 27±0.5° C., a relative humidity of 65-80% and a 12:12 hour photo period. The light period of (450 Lux) was set from 8:00 to 20:00. After hatching from the eggs, larvae were kept in water basins (30×30×10 cm) filled with a 1:1 mixture of deoxygenized tap and deionized water and fed with fish food flakes (Tetra Min®, Melle, Germany). Pupae were transferred to a cage (40×30×20 cm) for emergence, adult mosquitos were provided with sugar solution (10% dextrose). Mosquitos at an age of 7-12 days after emergence, that have never received a blood meal, were used for the cage tests.

Test Room.

Cage tests were performed in a climatized room of 41 m³ without windows. The temperature and relative humidity of the room air were set to 27±1° C. and 75±5% RH. The room was illuminated with full spectrum LED light tubes (intensity 450 Lux).

Test Cages.

Biogent (BG) test cages (Biogents AG, Regensburg, Germany) were used. These cages are an improved cage over conventional test cages for the evaluation of mosquito repellants (Obermayr et al., 2010). The cages have a volume of 27 cm³ (41×41×16 cm). Four sides of a cage are made of acrylic glass, the floor is made of metal sheet and the rear side is covered by a gauze sleeve. The floor sheet is equipped with a test window (size 56 cm²; 14.8×3.8 cm) for the exposure of the treated arm. In between tests, BG cages are connected to a ventilation system that provides it with clean, warm, and humid air (26±1° C., 75±10% RH) to remove remaining host odors and repellent volatiles from the air inside the cage.

Each cage was filled with populations of 30 mosquitos that were lured out of their rearing cages by a natural stimulus (human hand) to ensure that only host-seeking females are used for the repellent tests.

Test Procedure.

Prior to an individual efficacy test, the biting activity of the test mosquitos was verified with the untreated forearm of the volunteer. In order to keep the biting pressure on the untreated skin low, a modified spacer covered with fine mosquito netting was used during control tests. In this way, mosquitoes were still attracted to the skin odors and land on the net, however, they were unable to reach the skin and pierce it. The modified spacer was not used during the tests of the repellent treated skin in which mosquitoes were allowed to be in direct contact.

Positive Ae. aegypti biting activating requires a minimum of 10 landings in 30 seconds. If biting activity was low, 5 to 10 new mosquitoes were added to the cage or 30 fresh mosquitoes were used. The exact time until 10 landings was documented, and this time value was used for the calculation of the protective percentage on the treated arm. Discussed below.

Cage tests were performed following recommendations by two guidelines for repellent testing published by the American Protection Agency (EPA, 2010) and the World Health Organization (WHO, 2009).

Repellent efficacy was verified for the first time shortly after product application and then again in regular 30-minute intervals up to a maximum of 8 hours or until repellency failed. Each single test lasted 2 minutes, during this time the number of landings and bites on the treated skin were recorded.

Repellent efficacy was evaluated using: (1) the time until first bite (FB) and (2) the time until first confirmed bite (FCB=one bite followed by another one within the same test or within the consecutive test after 30 minutes). The FCB is defined to mark the end of complete protection time and is usually used as the criterion for break-off for repellent tests (according to the technical notes for guidance by the European Chemicals Agency, ECHA 2018). The WHO suggests using the first bite as criterion for break-off (WHO, 2009).

Tests were conducted with 3 volunteers (2 males, 26 and 32 years, 1 female, 21 years) against one mosquito species, the yellow fever mosquito Ae. aegypti. All volunteers were attractive to the test mosquito species, thereby meeting the requirements to participate in repellent efficacy studies.

Each volunteer received his or her own cage for testing, cages were never switched. Cages were connected to the air ventilation system in between single tests (zero control and repellent efficacy test) to avoid an accumulation of host odors and active ingredients inside the cage. Test mosquitoes that started to engorge blood during the test were replaced with new individuals to ensure that the number of host-seeking females stayed constant throughout the test day.

Data Analysis.

Mean times until first bite and FCB as well as corresponding standard deviations were calculated from data sets generated by three volunteers (n=3).

### Results.

Protection Times Against Ae. Aegpyti.

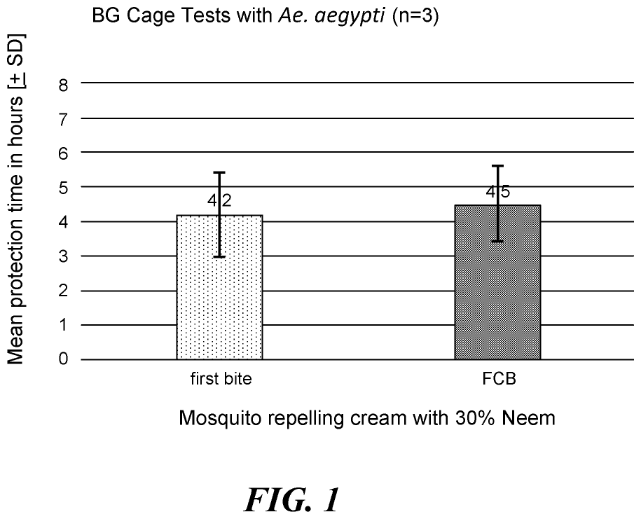

Test mosquitoes showed a reliable biting activity throughout the entire study, ten probings on untreated skin were recorded after an average of 22.7±6.1 seconds (n=30). Biting activity test results are shown in Table 7. Mean protection times until first bite and FCB are shown in Table 8 and FIG. 1. Raw data is shown in Table 9.

TABLE 7
Biting Activity Tests (untreated skin)

| | | Mean time (sec) | |
|---|---|---|---|
| Volunteer | n | until 10th probing | SD |
| 1, m 32 | 8 | 22.9 | 4.3 |
| 2, f 21 | 11 | 26.0 | 4.4 |
| 3, m 26 | 11 | 19.4 | 6.8 |

TABLE 8
Mean Protection Times in Cage with Ae. aegypti

| | Mean protection (h) | Mean protection (h) |
|---|---|---|
| Test formulation | until first bite (±SD) | until FCB (±SD) |
| Cream with 30% Neem | 4.2 ± 1.2 | 4.5 ± 1.1 |

TABLE 9
Raw Data

| Volunteer | First bite (h) | FCB (h) |
|---|---|---|
| 1, m 32 | 2.5 | 3 |
| 2, f 21 | 5 | 5.5 |
| 3, m 26 | 5 | 5 |

The mosquito repellent cream with 30% Neem provided an average protection of 4.2 hours from first bite and 4.5 hours from the FCB. FIG. 1 displays the protection times measured in cage tests.

The laboratory cage tests are the standard method to evaluate the contact- or bite-preventing potential of mosquito repellents. In contrast to field tests, which provide the most valuable information but are greatly influenced by a variety of abiotic and biotic factors (e.g., climatic conditions, mosquito population, mosquito density, activity patterns), laboratory cage tests can be performed at any time under standardized conditions and allow the use of laboratory-reared, pathogen-free vectors of diseases, which are important targets of personal protection measures.

Tests were performed with one mosquito species of medical importance. The diurnal yellow fever mosquito Ae. aegypti is very aggressive, has a broad activity pattern and can easily be maintained under laboratory conditions. Not only for these reasons this species is used as a standard mosquito for behavioral tests by research groups worldwide; Ae. aegypti is also the main vector of important arboviral diseases such as dengue and zika.

Cage tests were performed on the basis of two guidelines for repellent testing published by the EPA (2010) and the WHO (2009). The applied procedure is an improved version and introduces a few modifications to the conventional set-up in order to create more defined testing conditions and increase the reproducibility of the test (Obermayr et al. 2010). Cages were connected to an air ventilation system in-between single tests, by doing so the incoming warm and humid air prevented the accumulation of host odors and repellent substances inside the test cages. The use of a defined area on the forearm, instead of using the entire forearm, also minimized the entry of active substances into the test cage. Compared to conventional cages, test mosquitoes were exposed to fewer amounts of repellent for a shorter period of time which prevents exhaustion and a decrease in biting activity. EPA and WHO protocols use hundreds of mosquitoes per test cage to compensate for mosquito exhaustion throughout the test day, with the air ventilation- and test window system cages of the present experiments required only 30 females, which also reduced density-related stress. The required control biting rate of 10 bites (landings) in 30 seconds was also achieved with 30 test mosquitoes.

Mosquito-density in the test cage and short distances to the treatment area may still lead to a higher biting pressure compared to natural conditions in the field. Thus, shorter repellent protection times can occur during laboratory cage test studies. However, a comparison of present cage tests, conventional arm-in-cage tests and field tests revealed, that protection times obtained during present cage tests can be better related to field test data than protection times documented with conventional tests (Obermayr et al., 2010).

Cage tests were performed with 3 volunteers. During control tests of untreated skin, it took an average of 19.4 to 26 seconds until 10 probings by Ae. aegypti were counted. The biting activity tests/attractiveness of volunteers do not allow to draw conclusions on the protective effects provided by a repellent. Differences in individual protection times are more likely related to the skin properties of the volunteers (absorption & evaporation rates) and not so much to the individual attractiveness for the test mosquito species.

The present study aimed to estimate the protection times of a mosquito repelling cream containing 30% Neem. The results revealed that this cream provided an average protection of 4.2 hours from the first bite and an average of 4.5 hours from the FCB.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### REFERENCES

European Chemical Agency (2018) Guidance on the Biocidal Products Regulation. Volume II Efficacy—Assessment and Evaluation (Parts B&C). Version 3.0, April 2018.
United States Environmental Protection Agency (2008) Product Performance Test Guidelines. Insect Repellents to be Applied to Human Skin. 23 Sep. 2008.
Obermayr, U. et al. (2010) A Novel Test Cage with an Air Ventilation System as an Alternative to Conventional Cages for the Efficacy Testing of Mosquito Repellents. Journal of Medical Entomology 47(6):1116-1122.
World Health Organization (2009). Guidelines for Efficacy Testing of Mosquito Repellents for Human Skin.

1. A composition comprising up to about 40% neem, about 3% kokum butter, an antioxidant, an emulsifying agent and a preservative.

2. The composition of claim 1, wherein the neem is present in an amount of from about 1% to about 20%, by weight.

3. The composition of claim 1, wherein the neem is present in an amount of less than 10%, by weight.

4. The composition of claim 1, wherein the antioxidant is vitamin E and/or tocopherol.

5. The composition of claim 1, wherein the antioxidant is present in an amount of between about 0.01% and 5%, by weight.

6. The composition of claim 1, wherein the emulsifying agent is carbopol (Carbomer) 940, carbopol 934, carbopol 941, carbopol 1342 and gulf Polymer P18 (octadecene/maleic anhydride copolymer), a C₁₂-C₂₂ alkyl-substituted acrylic acid copolymer, or any combination thereof.

7. The composition of claim 1, wherein the emulsifying agent is present in an amount of between about 1% and 10%, by weight.

8. The composition of claim 1, wherein the preservative is methylparaben, propylparaben, benzyl alcohol, ascorbyl palmitate, ascorbic acid, or any combination thereof.

9. The composition of claim 1, wherein the preservative comprises phenoxyethanol, caprylyl glycol, sorbic acid, or any combination thereof.

10. The composition of claim 11, wherein the preservative is Optiphen PLUS or sorbic acid.

11. The composition of claim 1, wherein the preservative is present in an amount of about 0.01% and 5%, by weight.

12. The composition of claim 1, further comprising taro.

13. The composition of claim 12, wherein the taro is present in an amount of between about 0.1% to about 5%, by weight.

14. The composition of claim 1, further comprising a scent agent.

15. The composition of claim 14, wherein the scent agent is gardenia or cinnamon essential oil.

16. The composition of claim 1, wherein the composition is free or substantially free of deet, citronella, and equivalents or combinations thereof.

17. A composition comprising neem, taro, a fragrance, kokum butter, an antioxidant, a preservative, and an emulsifying agent.

18. A composition comprising neem, taro, gardenia, kokum butter, vitamin E, Optiphen PLUS, and ECOMulse or neem, kokum butter, cinnamon essential oil, fragrance, sorbic acid, and ECOMulse.

19. A composition comprising, on a weight basis, about 7% neem, about 2.5% taro, about 2.5% gardenia, and further comprising kokum butter, vitamin E, Optiphen PLUS, and ECOMulse.

20-22. (canceled)

23. The composition of claim 1, wherein the composition is a topical composition.

24-30. (canceled)
